(19) United States
(12) Patent Application Publication
(10) Pub. No.: US 2025/0115840 A1
(43) Pub. Date: Apr. 10, 2025

(54) CULTURE APPARATUS

(71) Applicants: Canon Kabushiki Kaisha, Tokyo (JP); CANON MEDICAL SYSTEMS CORPORATION, Tochigi (JP)

(72) Inventors: Shuhei IKEDA, Yokohama (JP); Tomoki HANEISHI, Kita-ku (JP)

(73) Assignees: Canon Kabushiki Kaisha, Tokyo (JP); CANON MEDICAL SYSTEMS CORPORATION, Tochigi (JP)

(21) Appl. No.: 18/905,983

(22) Filed: Oct. 3, 2024

(30) Foreign Application Priority Data
Oct. 6, 2023 (JP) 2023-174551

(51) Int. Cl. C12M 1/04; C12M 1/00; C12M 1/26

(52) U.S. Cl. CPC C12M 23/24; C12M 23/40; C12M 29/06; C12M 33/04

(57) ABSTRACT

A culture apparatus according to the present invention includes a housing, a supply unit, a discharge unit, and a gas permeation membrane. The supply unit is disposed inside the housing and supplies a culture vessel with a culture medium for culturing of cells. The discharge unit is disposed inside the housing and discharges liquid droplets for detaching the cells held in the culture vessel. The gas permeation membrane is disposed in the housing. With that, the culture apparatus according to the present embodiment enables performing cell culturing, cell detachment, and gas exchange in the closed condition.

### CROSS-REFERENCE TO RELATED APPLICATIONS

This application is based upon and claims the benefit of priority from Japanese Patent Application No. 2023-174551, filed on Oct. 6, 2023; the entire contents of which are incorporated herein by reference.

### FIELD

Embodiments described herein relate generally to a culture apparatus.

### BACKGROUND

There are two types of cell culturing, namely, open-type culturing and closed-type culturing. The open-type culturing is a commonly-used culturing method in which, usually, culturing is performed using a dish representing a culture vessel, and tasks such as culture medium replacement and passage culture are performed by the worker by opening the lid of the dish. In the open-type culturing, the space used for cell culturing is basically in the open condition. Hence, the worker is highly likely to get exposed to bacterial contamination; and, in the case of using a virus vector, there is a high infection risk to the worker. On the other hand, in the closed-type culturing, culturing is performed in a sealed manner so as to ensure that there is no leakage of the liquid from the inside of the main body of the culture vessel and that there is no mixing of any liquid from outside. Thus, regarding the culturing performed in a closed-type (sealed) culture vessel, the risk of bacterial contamination during the culturing is exceedingly small. However, since the tasks such as culture medium replacement and cell detachment are performed by the worker in an open condition, there is a risk of infection. For that reason, there is a demand for a culture apparatus that enables performing culture medium replacement and cell detachment too in a closed condition.

For example, in the case of collecting iPS cells, usually, water droplets are applied onto the cells using a micropipette and the cells are detached from the vessel; and then the suspension liquid is aspirated using an aspirator and the cells are collected. However, this method is difficult to implement inside a closed-type vessel. Hence, it becomes necessary to implement a different detachment method than a commonly-used method. Alternatively, there is a method in which, in the closed condition, the vessel is agitated so as to apply the shearing force to the cells. However, it is not possible to detach cells having strong adherence strength, such as the iPS cells.

Moreover, in the case of using a closed-type (sealed) culture vessel, the culturing performed in the culture vessel requires gas exchange. Hence, when cell culturing or cell detachment is performed in the closed condition, it becomes necessary to perform gas exchange that is required for the culturing in the culture vessel.

### BRIEF DESCRIPTION OF THE DRAWINGS

FIG. 1 is a perspective view illustrating a configuration of a culture apparatus according to an embodiment;

FIG. 2 is a lateral cross-sectional view illustrating a configuration of the culture apparatus according to the present embodiment;

FIG. 3A is a perspective view of a second housing portion in the culture apparatus according to the present embodiment, and represents a diagram for explaining an example of a gas permeation membrane;

FIG. 3B is a side view of the second housing portion in the culture apparatus according to the present embodiment, and represents a diagram for explaining an example of the gas permeation membrane; and

FIG. 4 is a side view of the culture apparatus according to the present embodiment, and represents an enlarged view of the portion in which the gas permeation membrane is disposed.

### DETAILED DESCRIPTION

A culture apparatus according to an embodiment includes a housing, a supply unit, a discharge unit, and a gas permeation membrane. The supply unit is disposed inside the housing and supplies a culture vessel with a culture medium for culturing of cells. The discharge unit is disposed inside the housing and discharges liquid droplets for detaching the cells held in the culture vessel. The gas permeation membrane is disposed in the housing.

An exemplary embodiment of a culture apparatus is described below in detail with reference to the accompanying drawings. However, the embodiments are not limited to the embodiment described below. Moreover, the details given in a single embodiment are applicable in principle to other embodiments too in an identical manner.

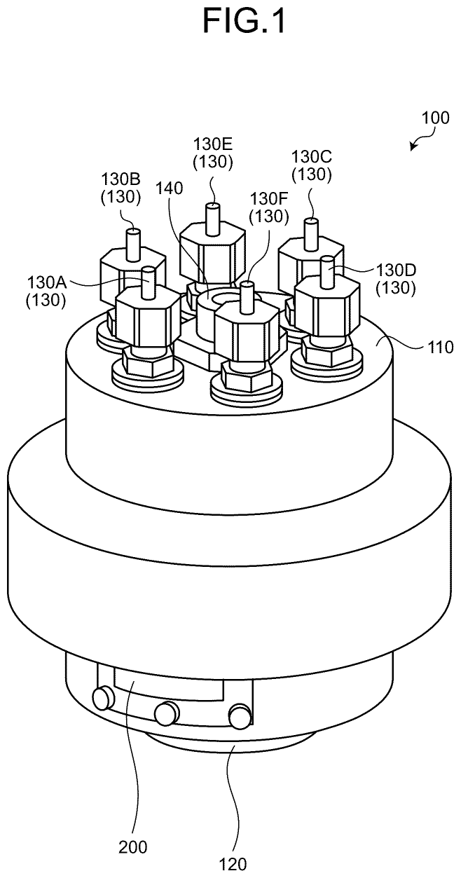

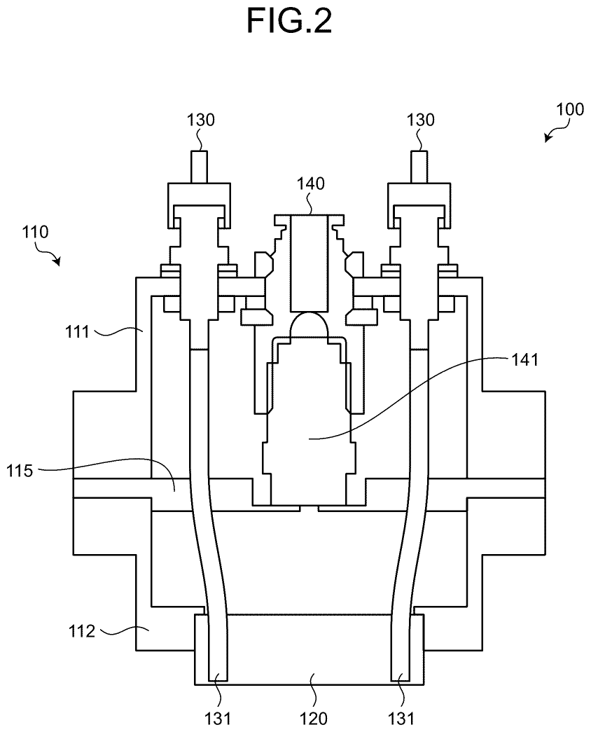

FIG. 1 is a perspective view illustrating a configuration of a culture apparatus 100 according to the present embodiment. FIG. 2 is a lateral cross-sectional view illustrating a configuration of the culture apparatus 100 according to the present embodiment.

As illustrated in FIG. 1, the culture apparatus 100 according to the present embodiment includes a housing 110, a light shielding plate 115, and a plurality of couplings 130.

The housing 110 has a cylindrical portion in which tubes (explained later) are disposed; and includes a first housing portion 111 and a second housing portion 112 that is disposed in the lower part of the first housing portion 111. The light shielding plate 115 is disposed in between the first housing portion 111 and the second housing portion 112. In the culture apparatus 100 according to the present embodiment, an arbitrary sealing mechanism is disposed in between the first housing portion 111 and the light shielding plate 115 as well as in between the light shielding plate 115 and the second housing portion 112. As a result, the sealing property of the housing 110 is secured.

The first housing portion 111, the light shielding plate 115, and the second housing portion 112 have, for example, screw holes formed thereon in the vertical direction and are screw-clamped together. That is, the first housing portion 111 and the second housing portion 112 support the light shielding plate 115.

In the culture apparatus 100 according to the present embodiment, in order to reduce the infection risk to the worker, tubes are used for the purpose of culture medium replacement and cell collection in the closed condition.

In the upper part of the first housing portion 111, the plurality of couplings 130 are disposed. As illustrated in FIG. 2, the culture apparatus 100 according to the present embodiment further includes a plurality of tubes 131. Each of the plurality of tubes 131 is disposed inside the housing 110 via one of the plurality of couplings 130.

For example, the plurality of tubes 131 include a tube for cell seeding and culture medium replacement, a tube for waste solution collection, a tube for PBS addition, a tube for EDTA addition, a tube for cell suspension liquid collection, and a spare tube. In the example illustrated in FIG. 1, the plurality of couplings 130 include a coupling 130 that is connected to the tube 131 for cell seeding and culture medium replacement; a coupling 130B that is connected to the tube 131 for waste solution collection; a coupling 130C that is connected to the tube 131 for PBS addition; a coupling 130D that is connected to the tube 131 for EDTA addition; a coupling 130E that is connected to the tube 131 for cell suspension liquid collection; and a coupling 130F that is connected to the spare tube 131.

As illustrated in FIG. 2, inside the housing 110, a plurality of tubes 131 is disposed. On the light shielding plate 115, openings are formed through which the plurality of tubes 131 are disposed.

As illustrated in FIG. 2, in the bottom portion of the second housing portion 112, a culture vessel 120 is disposed that represents a dish for culturing cells. The bottom face of the culture vessel 120 has a circular shape. For example, the bottom face of the culture vessel 120 has the diameter equal to or greater than 35 mmϕ and equal to or smaller than 100 mmϕ. On top of the culture vessel 120, the culture apparatus 100 according to the present embodiment is placed as a lid. Regarding the height of the culture apparatus 100, for example, the height from the bottom face of the culture vessel 120 to the leading end of the plurality of couplings 130 is equal to or greater than 30 mm and equal to or shorter than 120 mm. Inside the culture vessel 120, the leading ends of the plurality of tubes 131 are disposed.

In the culture apparatus 100 according to the present embodiment, a nozzle is used for performing cell detachment in the closed condition.

As illustrated in FIGS. 1 and 2, the culture apparatus 100 according to the present embodiment further includes a coupling 140. Moreover, as illustrated in FIG. 2, the culture apparatus 100 according to the present embodiment further includes a detachment nozzle 141.

The coupling 140 is disposed in the central portion of the upper part of the first housing portion 111. The detachment nozzle 141 is disposed inside the housing 110 and is connected to the coupling 140 in the upper part of the first housing portion 111. In the central portion of the light shielding plate 115, an opening is formed into which the leading end of the detachment nozzle 141 is disposed. The distance between the leading end of the detachment nozzle 141 and the bottom face of the culture vessel 120 is, for example, equal to or greater than 10 mm and equal to or shorter than 50 mm.

The culture vessel 120 has a circular bottom face. Hence, the detachment nozzle 141 discharges, in a conical manner, liquid droplets for detaching the cells held in the culture vessel 120. For example, the detachment nozzle 141 sprays liquid droplets in the form of mist. For example, the detachment nozzle 141 applies liquid droplets onto 95% or more of the base area of the culture vessel 120. As a result, it becomes possible to detach the cells with ease.

The following explanation is given about the role of the light shielding plate 115.

For example, the worker observes the inside of the culture vessel 120 using a camera. The light shielding plate 115 that is disposed inside the housing 110 is disposed to enable observation using a camera; and, when the culture vessel 120 is illuminated from the side, is used to prevent the detachment nozzle 141 and the tubes 131 from being photographed.

The following explanation is given about the role of the plurality of tubes 131 and the detachment nozzle 141.

In the culture apparatus 100 according to the present embodiment, the detachment nozzle 141 is used because no lid is provided for enabling culture medium replacement and cell detachment in the closed condition. In the culture apparatus 100 according to the present embodiment, for example, phosphate-buffered saline (PBS) is sprayed as the liquid droplets from the detachment nozzle 141; and the cells adhered to the bottom of the culture vessel 120 are detached, and the detached cells are sucked in and collected through the tube 131 for cell suspension liquid collection.

More particularly, in the culture apparatus 100 according to the present embodiment, firstly, a process is performed in which cells are seeded in the culture vessel 120 using the tube 131 for cell seeding and culture medium replacement. Then, in the culture apparatus 100 according to the present embodiment, a process is performed in which the culture medium in the culture vessel 120 is replaced using the tube 131 for cell seeding and culture medium replacement and the tube 131 for waste solution collection. Since the cells inside the culture vessel 120 are unclean due to the waste matter such as the culture medium, in the culture apparatus 100 according to the present embodiment, a process is performed in which the cells inside the culture vessel 120 are cleaned by adding PBS as a cleaning fluid using the tube 131 for PBS addition, and then the PBS is collected using the tube 131 for waste solution collection. Subsequently, in the culture apparatus 100 according to the present embodiment, a process is performed in which the adhesion force among the cells and the adhesion force between the cells and the culture vessel 120 is weakened by adding ethylenediaminetetraacetic acid (EDTA) as a liquid solution into the culture vessel 120 using the tube 131 for EDTA addition, and then the EDTA is collected using the tube 131 for waste solution collection. Subsequently, in the culture apparatus 100 according to the present embodiment, a process is performed in which PBS liquid droplets are sprayed into the culture vessel 120 using the detachment nozzle 141, so that the water droplets are applied inside the culture vessel 120 and the cells adhered to the bottom of the culture vessel 120 are detached. Lastly, in the culture apparatus 100 according to the present embodiment, a process is performed in which the cells that have been detached inside the culture vessel 120 are sucked in and collected using the tube 131 for cell suspension liquid collection. At that time, a culture medium is added using the tube 131 for medium replacement, and the added culture medium and the cells are collected using the tube 131 for waste solution collection and the tube 131 for cell suspension liquid collection. All the abovementioned processes are performed in the closed condition.

Meanwhile, the tube 131 for cell seeding and medium replacement represents an example of a “supply unit”. The tube 131 for waste solution collection represents an example of a “waste solution collection unit”. The detachment nozzle 141 represents an example of a “discharge unit”. The tube 131 for cell suspension liquid collection represents an example of a “cell collection unit”.

In the case of using a closed-type (sealed) culture vessel 120, the culturing performed in the culture vessel 120 requires gas exchange. Moreover, although PBS is sprayed using the detachment nozzle 141, it is desirable to vent the gas to the outside of the culture vessel 120 in order to hold down the pressure loss. For that reason, in the culture apparatus 100 according to the present embodiment, in the case of performing medium replacement and cell detachment in the closed condition, it becomes necessary to perform gas exchange that is required for the culturing in the culture vessel 120 and to vent the gas to the outside of the culture vessel 120.

In that regard, in order to enable cell culturing, cell detachment, and gas exchange in the closed condition, the culture apparatus 100 according to the present embodiment includes the housing 110, the supply unit (the tube 131 for cell seeding and medium replacement), the discharge unit (the detachment nozzle 141), and a gas permeation membrane 200 illustrated in FIG. 1. The supply unit is disposed inside the housing 110 and supplies the culture vessel 120 with a culture medium for cell culturing. The discharge unit is disposed inside the housing 110 and discharges the liquid droplets that detach the cells held in the culture vessel 120. The gas permeation membrane 200 is disposed inside the housing 110 and performs gas exchange required for the culturing in the culture vessel 120 and discharges the gas to the outside of the culture vessel 120.

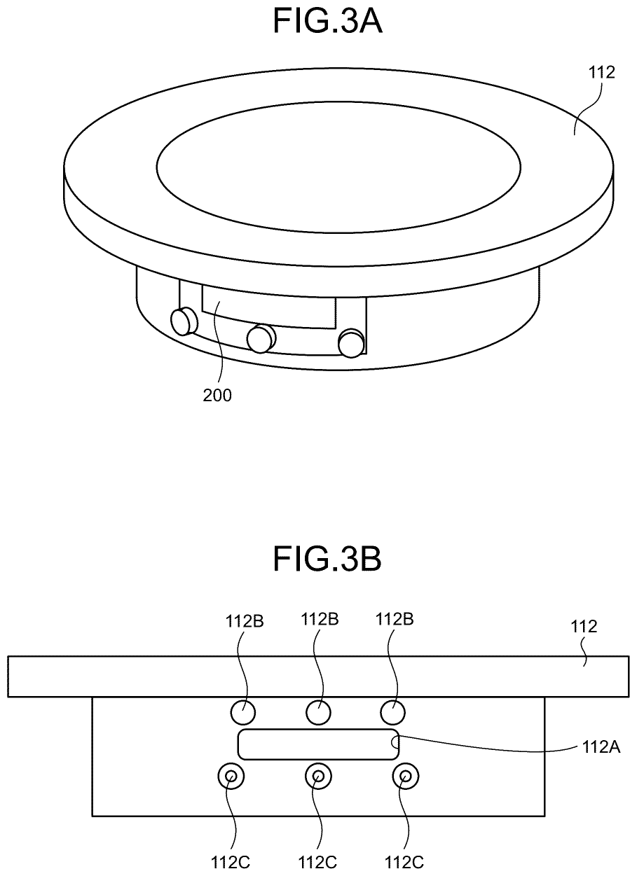

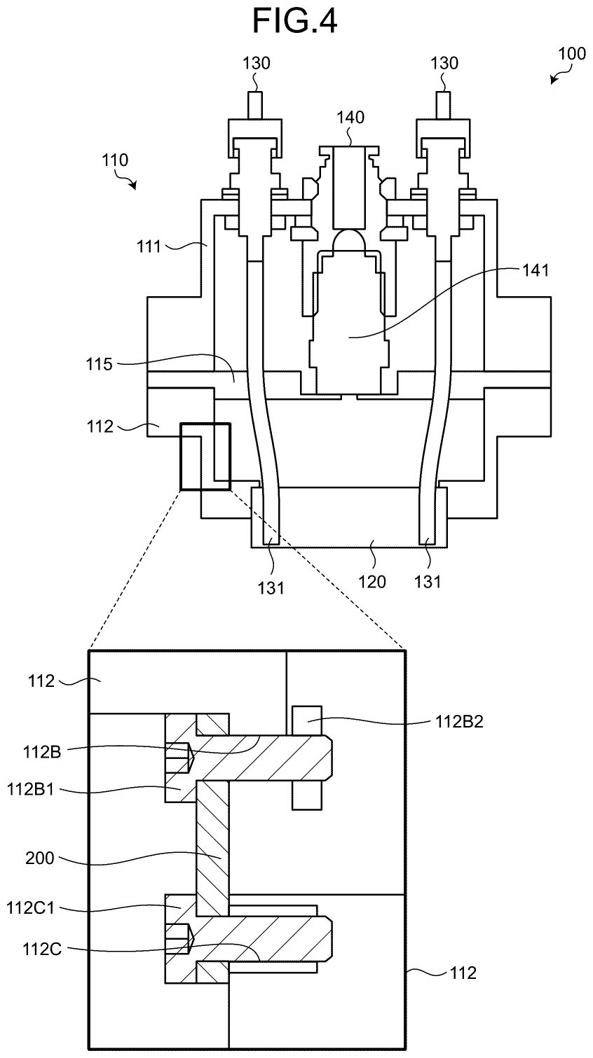

FIG. 3A is a perspective view of the second housing portion 112 in the culture apparatus 100 according to the present embodiment, and represents a diagram for explaining an example of the gas permeation membrane 200. FIG. 3B is a side view of the second housing portion 112 in the culture apparatus 100 according to the present embodiment, and represents a diagram for explaining an example of the gas permeation membrane 200. FIG. 4 is a side view of the culture apparatus 100 according to the present embodiment, and represents an enlarged view of the portion in which the gas permeation membrane 200 is disposed.

The gas permeation membrane 200 is disposed in between the detachment nozzle 141 and the culture vessel 120 in the housing 110. For example, as illustrated in FIG. 1, the gas permeation membrane 200 is disposed on the outer periphery of the culture vessel 120 in the housing 110. More particularly, the outer periphery of the housing 110 is larger than the outer periphery of the culture vessel 120 and, as illustrated in FIG. 3A, the gas permeation membrane 200 is disposed on the outer periphery of the cylindrical portion of the second housing portion 112 of the housing 110 on which the tubes 131 are also disposed.

As illustrated in FIG. 3B, in the second housing portion 112, an opening 112A is formed with which the gas permeation membrane 200 is covered. Moreover, on the outer periphery of the second housing portion 112, screw holes 112B and 111C are disposed for the purpose of attaching the gas permeation membrane 200. The screw holes 112B are disposed on the upper side of the opening 112A, and the screw holes 112C are disposed on the lower side of the opening 112A.

The gas permeation membrane 200 is abutted against the opening 112A illustrated in FIG. 3B. Then, as illustrated in FIG. 4, a screw 112B1 that is inserted into the corresponding screw hole 112B from the outside of the second housing portion 112 is screw-clamped with a nut 112B2 that is placed from the inside of the second housing portion 112 and that has the screw thread formed on the inner face of the hole thereof. Furthermore, a screw 112C1 that is inserted into the corresponding screw hole 112C from the outside of the second housing portion 112 is screw-clamped with the same screw hole 112C that has the screw thread formed thereon. For example, the gas permeation membrane 200 has a membrane filter as the member. The membrane filter is attached to the second housing portion 112 with the aim of preventing a decline in the flow rate attributed to the pressure loss at the time of performing gas exchange during culturing and at the time of spraying liquid droplets from the detachment nozzle 141.

For example, if the area of the membrane filter is increased, the supply of carbon dioxide (CO2) becomes easier and the hydrogen-ion concentration (pH) can be maintained. However, the volatilization volume increases and there is a risk of poor growth of cells. The gas exchange capacity and the volatilization volume are in a trade-off relationship, and the area of the membrane filter has an optimum value. In terms of gas exchange efficiency, it is desirable that the membrane filter is positioned close to the bottom face of the culture vessel 120; and, from the perspective of spraying liquid droplets, it is desirable that the liquid droplets do not come in contact with the membrane filter. For that reason, it is desirable that the distance between the gas permeation membrane 200 and the culture vessel 120 is equal to or shorter than 50 mm. For example, regarding the vertical distance as well as the horizontal distance between the gas permeation membrane 200 and the culture vessel 120, it is desirable that the distance is equal to or shorter than 50 mm.

Moreover, it is desirable that the average pore diameter of the gas permeation membrane 200 is such that sufficient aeration (CO2 gas exchange) is possible for performing cell culturing and that neither the liquid inside the culture vessel oozes out nor any outside liquid gets mixed. For example, it is desirable that the average pore diameter of the gas permeation membrane 200 is equal to or smaller than 0.2 μm.

As explained above, in the culture apparatus 100 according to the present embodiment, inside the housing 110, the supply unit (the tube 131 for cell seeding and medium replacement) supplies the culture vessel 120 with a culture medium for cell culturing, and the discharge unit (the detachment nozzle 141) discharges liquid droplets that detach the cells held in the culture vessel 120. Moreover, in the culture apparatus 100 according to the present embodiment, the gas permeation membrane 200 that is disposed in the housing 110 performs gas exchange required for the culturing in the culture vessel 120 and vents the gas to the outside of the culture vessel 120. As a result, the culture apparatus 100 according to the present embodiment enables performing cell culturing, cell detachment, and gas exchange in the closed condition, and enables achieving reduction in the infection risk to the worker.

According to at least one embodiment described above, it becomes possible to perform cell culturing, cell detachment, and gas exchange in the closed condition.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A culture apparatus comprising:
a housing;
a supply unit that is disposed inside the housing and that supplies a culture vessel with a culture medium for culturing of cells;
a discharge unit that is disposed inside the housing and that discharges liquid droplets for detaching the cells held in the culture vessel; and
a gas permeation membrane that is disposed in the housing.

2. The culture apparatus according to claim 1, wherein the gas permeation membrane is disposed in between the discharge unit and the culture vessel in the housing.

3. The culture apparatus according to claim 1, wherein the gas permeation membrane is disposed on outer periphery of the culture vessel in the housing.

4. The culture apparatus according to claim 1, wherein distance between the gas permeation membrane and the culture vessel is equal to or shorter than 50 mm.

5. The culture apparatus according to claim 1, wherein bottom face of the culture vessel is circular in shape and has diameter equal to or greater than 35 mmϕ and equal to or smaller than 100 mmϕ.

6. The culture apparatus according to claim 1, wherein height of the culture apparatus is equal to or greater than 30 mm and equal to or shorter than 120 mm.

7. The culture apparatus according to claim 1, wherein average pore diameter of the gas permeation membrane is equal to or smaller than 0.2 μm.

8. The culture apparatus according to claim 1, further comprising:
a waste solution collection unit that collects the culture medium supplied to the culture vessel; and
a cell collection unit that collects the cells detached in the culture vessel, wherein
the supply unit also seeds the cells in the culture vessel, and
a process by which the supply unit seeds the cells in the culture vessel, a process by which the supply unit and the waste solution collection unit perform replacement of the culture medium in the culture vessel, a process by which the discharge unit detaches the cells inside the culture vessel, and a process by which the cell collection unit collects the cells inside the culture vessel are performed in a closed condition.

9. The culture apparatus according to claim 1, wherein distance between the discharge unit and bottom face of the culture vessel is equal to or greater than 10 mm and equal to or shorter than 50 mm.

10. The culture apparatus according to claim 1, wherein
bottom face of the culture vessel is circular in shape, and
the discharge unit discharges the liquid droplets in a conical manner.

11. The culture apparatus according to claim 1, wherein the discharge unit applies the liquid droplets onto 95% or more of base area of the culture vessel.

12. The culture apparatus according to claim 1, wherein the gas permeation membrane has a membrane filter as a member.
